(19) United States
(12) Patent Application Publication
(10) Pub. No.: US 2022/0348900 A1
(43) Pub. Date: Nov. 3, 2022

(54) PROCESSES FOR PURIFYING DOWNSTREAM PRODUCTS OF IN VITRO TRANSCRIPTION

(71) Applicant: ModernaTX, Inc., Cambridge, MA (US)

(72) Inventors: Michael Shamashkin, Cambridge, MA (US); Matthew Scott, Cambridge, MA (US); Peter Wojciechowski, Cambridge, MA (US)

(73) Assignee: ModernaTX, Inc., Cambridge, MA (US)

(21) Appl. No.: 17/634,939

(22) PCT Filed: Aug. 13, 2020
(86) PCT No.: PCT/US2020/046069

§ 371 Date: Feb. 11, 2022

Related U.S. Application Data
(60) Provisional application No. 62/886,840, filed on Aug. 14, 2019.

(51) Int. Cl. C12N 15/10; B01D 15/32; C12Q 1/6806; B01J 20/26; B01J 20/28

(52) U.S. Cl. CPC C12N 15/101; B01D 15/327; C12Q 1/6806; B01J 20/264; B01J 20/28085

(57) ABSTRACT

Provided herein, in some embodiments, are methods of purifying low-salt RNA compositions using denaturing oligo-dT chromatography.

### RELATED APPLICATIONS

This application claims the benefit under 35 U.S.C. § 119(e) of U.S. provisional application No. 62/886,840, filed Aug. 14, 2019, the content of which is incorporated by reference herein in its entirety.

### BACKGROUND

In vitro transcription (IVT) uses bacteriophage DNA-dependent ribonucleic acid (RNA) polymerases (e.g., SP6, T3 and T7) to synthesize template-directed messenger RNA (mRNA) transcripts. Problems in an IVT reaction can result in complete failure (e.g., no transcript generated) or in transcripts that are the incorrect size (e.g., shorter or longer than expected), for example. Specific problems associated with IVT reactions include, for example, abortive (truncated) transcripts, run-on transcripts, poly-A tail variants/3′ heterogeneity (including low percent poly-A tailed mRNA), mutated transcripts, and/or double-stranded contaminants produced during the reactions. One mechanism to counteract these problems resulting from IVT reactions is to purify the mRNA products after the reaction is complete.

### SUMMARY

The present disclosure provides, in some embodiments, methods of isolating a high yield of highly pure ribonucleic acid (RNA), such as messenger RNA (mRNA), for example, from an in vitro transcription (IVT) reaction. Previous mRNA purifications have centered on the use of ambient oligo-dT alone or in combination with reverse-phase HPLC. However, these purification methods provide low percent tailed mRNA purity (ambient oligo-dT alone) or are cost-prohibitive at large scale (ambient oligo-dT in combination with reverse-phase HPLC). As such, new mRNA purification methods are needed. Surprisingly, studies herein show that in-line mixing of a high-salt buffer with a low-salt composition comprising denatured RNA significantly increases the relative yield of mRNA containing a poly-A tail. In some embodiments, at least 95% of the mRNA isolated using the methods provided herein has a poly-A tail; this percentage of poly-A-tailed RNA species is higher than the percentage purified using conventional RNA purification methods, such as reverse phase chromatography. Without being bound by theory, it is thought that denaturing the RNA before rapid in-line mixing with a high-salt buffer facilitates the removal of impurities associated with the RNA and facilitates highly selective binding of the RNA to an oligo dT resin. Further, the methods provided herein, in some embodiments, are easily scalable (e.g., purifications using columns with column volumes of at least 1 liter) and cost-effective.

Thus, aspects of the present disclosure provide methods that comprise in-line mixing a composition comprising denatured RNA with a high-salt buffer to produce a composition comprising denatured RNA and salt (e.g., at a concentration of at least 50 mM), binding the denatured RNA of the composition to an oligo-dT resin (e.g., at a temperature of lower than 40° C.), and eluting RNA from the oligo-dT resin (e.g., at least 95% of which is mRNA with a poly-A tail).

In some embodiments, the methods comprise (a) desalting a mixture (e.g., an IVT reaction mixture) comprising RNA to produce a low-salt RNA composition having a salt concentration of less than 20 mM, (b) heating the low-salt RNA composition to a temperature of higher than 60° C. to produce denatured RNA, (c) in-line mixing the composition comprising denatured RNA with a high-salt buffer to produce a composition comprising denatured RNA and salt at a concentration of at least 50 mM, (d) binding the denatured RNA of the composition produced in (c) to an oligo-dT resin at a temperature of lower than 40° C., and (e) eluting RNA from the oligo-dT resin.

In some embodiments, the methods comprise (a) desalting a mixture (e.g., an IVT reaction mixture) comprising RNA to produce a low-salt RNA composition having a conductivity of less than 2 mS/cm, (b) heating the low-salt RNA composition to a temperature of higher than 60° C. to produce denatured RNA, (c) in-line mixing the composition comprising denatured RNA with a high-salt buffer to produce a composition comprising denatured RNA and a conductivity of at least 5 mS/cm, (d) binding the denatured RNA of the composition produced in (c) to an oligo-dT resin at a temperature of lower than 40° C., and (e) eluting RNA from the oligo-dT resin. In some embodiments, the mixture comprising RNA is a diluted in vitro transcription (IVT) reaction. Other mixtures comprising RNA may be used.

In some embodiments, the salt of a high-salt buffer comprises sodium chloride (NaCl). Other salts may be used. In some embodiments, a high-salt buffer has a salt concentration of 100 mM to 1000 mM. For example, a high-salt buffer may have a salt concentration of 50 mM to 450 mM, 50 mM to 400 mM, 50 mM to 350 mM, 50 mM to 300 mM, 50 mM to 250 mM, 50 mM to 200 mM, 50 mM to 150 mM, 50 mM to 100 mM, 100 mM to 500 mM, 100 mM to 450 mM, 100 mM to 400 mM, 100 mM to 350 mM, 100 mM to 300 mM, 100 mM to 250 mM, 100 mM to 200 mM, 100 mM to 150 mM, 150 mM to 500 mM, 150 mM to 450 mM, 150 mM to 400 mM, 150 mM to 350 mM, 150 mM to 300 mM, 150 mM to 250 mM, 150 mM to 200 mM, 200 mM to 500 mM, 200 mM to 450 mM, 200 mM to 400 mM, 200 mM to 350 mM, 200 mM to 300 mM, or 200 mM to 250 mM. In some embodiments, a high-salt buffer has a conductivity of 5 mS/cm to 85 mS/cm. For example, a high-salt buffer may have a conductivity of 5 mS/cm to 10 mS/cm, 5 mS/cm to 15 mS/cm, 5 mS/cm to 25 mS/cm, 5 mS/cm to 35 mS/cm, 5 mS/cm to 50 mS/cm, 5 mS/cm to 60 mS/cm, 5 mS/cm to 70 mS/cm, 10 mS/cm to 20 mS/cm, 15 mS/cm to 25 mS/cm, 20 mS/cm to 30 mS/cm, 25 mS/cm to 35 mS/cm, 30 mS/cm to 40 mS/cm, 35 mS/cm to 45 mS/cm, 40 mS/cm to 50 mS/cm, 45 mS/cm to 55 mS/cm, 50 mS/cm to 60 mS/cm, 55 mS/cm to 65 mS/cm, 60 mS/cm to 70 mS/cm, 65 mS/cm to 75 mS/cm, 70 mS/cm to 80 mS/cm, or 75 mS/cm to 85 mS/cm.

In some embodiments, desalting comprises binding the RNA from a crude mixture to a hydrophobic interaction chromatography (HIC) resin and eluting the RNA from the HIC resin with low-salt buffer to produce the low-salt RNA composition. In some embodiments, the HIC resin is a (poly)styrene-divinylbenzene (PS-DVB) R150 bead resin (e.g., with 2000 Angstrom pores).

In some embodiments, a heating step(s) occurs for (is implemented for) 1 minute or less, or less than 1 minute (e.g., 10-60 seconds (s), 10-50 s, 10-40 s, 10-30 s, 10-20 s, 20-60 s, 20-50 s, 20-40 s, 20-30 s, 30-60 s, 30-50 s, 30-40 s, 40-60 s, 40-50 s, or 50-60 s). In some embodiments, a heating step(s) occurs for (is implemented for) at least 10 seconds.

In some embodiments, a heating step(s) occurs at (is implemented at) a temperature of 60° C. to 90° C. (e.g., 60° C. to 80° C., 60° C. to 70° C., 65° C. to 90° C., 65° C. to 80° C., 65° C. to 70° C., 70° C. to 90° C., 70° C. to 80° C., 75° C. to 90° C., 75° C. to 80° C., 80° C. to 90° C., or 85° C. to 90° C.).

In some embodiments, the secondary structure of RNA is monitored during a heating step (e.g., a heating step intended to denature RNA) using, for example, ultraviolet detection. In some embodiments, denaturation of RNA during a heating step (e.g., a heating step intended to denature RNA) is monitored using, for example, ultraviolet detection. In some embodiments, denaturation of RNA is monitored by collecting ultraviolet measurements of the hyperchromicity of the RNA, for example, before and after a heating step (e.g., a heating step intended to denature RNA).

In some embodiments, at least 90% (e.g., at least 91%, at least 92%, at least 93%, at least 94%, at least 95%, at least 96%, at least 97%, at least 98%, at least 99%, or 100%) of the total RNA in a composition comprises denatured RNA.

In some embodiments, a low-salt RNA composition is heated in the presence of a denaturant molecule, such as dimethyl sulfoxide, guanidine, or urea.

In some embodiments, in-line mixing of the composition comprising denatured RNA with a high-salt buffer occurs for (is implemented for) 1 minute or less, or less than 1 minute (e.g., 10-60 seconds (s), 10-50 s, 10-40 s, 10-30 s, 10-20 s, 20-60 s, 20-50 s, 20-40 s, 20-30 s, 30-60 s, 30-50 s, 30-40 s, 40-60 s, 40-50 s, or 50-60 s). In some embodiments, in-line mixing of the composition comprising denatured RNA with a high-salt buffer occurs before contacting the dT resin. In some embodiments, in-line mixing of the composition comprising denatured RNA with a high-salt buffer occurs concurrently with (e.g., at the same time) contacting composition with the dT resin. Without being bound by theory, it is thought that in-line mixing for a short period of time prevents the denatured RNA from folding and/or associating with impurities before contacting the dT resin.

In some embodiments, in-line mixing comprises in-line cooling of the composition comprising denatured RNA to a temperature of lower than 60° C. (e.g., lower than 60° C., 55° C., 50° C., 45° C., 40° C., 35° C., 30° C., 25° C., 20° C., 15° C., 10° C., or 5° C.). In some embodiments, in-line mixing comprises in-line cooling of the composition comprising denatured RNA to a temperature of lower than 60° C. but higher than 4° C. In some embodiments, a composition comprising denatured RNA is stored in a break tank following denaturation. In some embodiments, a composition comprising denatured RNA is stored in a break tank following denaturation and in-line cooling. In some embodiments, a composition comprising denatured RNA is stored in a break tank for 1-5 days (e.g., 1, 2, 3, 4 or 5 days) at 2-8° C. (e.g., 2° C., 3° C., 4° C., 5° C., 6° C., 7° C., or 8° C.).

In some embodiments, binding of the denatured RNA of the composition to an oligo-dT resin occurs at (is implemented at) a temperature of 4° C. to 25° C. (e.g., 4° C. to 20° C., 4° C. to 15° C., or 4° C. to 10° C.).

In some embodiments, the oligo-dT resin is a (poly)styrene-divinylbenzene (PS-DVB) bead resin (e.g., with 2000 Angstrom pores, e.g., derivatized with poly dT).

In some embodiments, binding of the denatured RNA of the composition to an oligo-dT resin occurs for (is implemented for) 20 minutes or less, or less than 20 minutes (e.g., 5 minutes (min) to 20 min, 5 min to 15 min, 5 min to 10 min, 10 min to 20 min, 10 min to 15 min, or 15 min to 20 min).

In some embodiments, the RNA eluted from the oligo-dT resin comprises at least 90% poly-A tailed mRNA. For example, the RNA eluted from the oligo-dT resin may comprise at least 91%, at least 92%, at least 93%, at least 94%, at least 95%, at least 96%, at least 97%, at least 98%, at least 99%, or 100% poly-A tailed mRNA. In some embodiments, the RNA eluted from the oligo-dT resin comprises at least 95% poly-A tailed mRNA.

### BRIEF DESCRIPTION OF THE DRAWINGS

FIG. 1 depicts a schematic of an example of a method for purifying mRNA using denaturing oligo-dT resin.

FIG. 2 depicts graphs showing melting curves of RNA molecules of two different lengths in the presence of varying salt concentrations.

FIG. 3 depicts a schematic of an example of an apparatus for purifying RNA using denaturing dT chromatography.

FIG. 4 depicts a graph showing the effect of different purification strategies of an IVT reaction on RNA purity, as assessed by percent poly-A tailed mRNA.

FIG. 5 depicts a schematic of examples of processes for purifying RNA produced by an IVT reaction.

FIG. 6 depicts a representative chromatogram of mRNA purified using hydrophobic interaction chromatography (HIC).

### DETAILED DESCRIPTION

In vitro transcription (IVT) reactions present a powerful platform for the production of RNA (e.g., mRNA). Nonetheless, in addition to producing the desired RNA product(s), IVT reactions also generate significant levels of impurities. In part because of those impurities, purification of the desired RNA product(s) has proved to be a challenge. Provided herein, in some embodiments, are methods for efficient, cost-effective removal of RNA impurities from large-scale IVT reactions. These methods, which include a desalting process, such as hydrophobic interaction chromatography (HIC), with RNA denaturation, enable high-yield isolation of a highly pure mRNA population. Unexpectedly, an in-line (continuous blend) mixing process that was incorporated into the methods further facilitated high-affinity binding of denatured mRNA to an oligo-dT resin. The in-line mixing process, which allowed for rapid mixing of the denatured RNA with a high-salt solution, was found to be critical in preventing the denatured RNA from associating (e.g., hybridizing) with impurities while also ensuring that the composition of denatured RNA comprised an optimal high salt concentration that maximizes binding of the denatured RNA to the oligo-dT resin.

Thus, as described herein, the present disclosure provides methods of purifying mixtures comprising RNA (e.g., mRNA produced by an IVT reaction). In some aspects, the methods comprise in-line mixing of a high-salt buffer with a low-salt denatured RNA composition that comprises denatured ribonucleic acid (RNA) to produce a high-salt composition comprising denatured RNA; and subsequently (e.g., immediately) binding the denatured RNA to an oligo-dT resin. In other aspects, the methods comprise (a) desalting a mixture comprising ribonucleic acid (RNA) to produce a low-salt RNA composition having a salt concentration of less than 20 mM; (b) heating the low-salt RNA composition to a temperature of higher than 60° C. to produce denatured RNA; (c) in-line mixing the composition comprising denatured RNA with a high-salt buffer to produce a composition comprising denatured RNA and salt at a concentration of at least 50 mM; (d) binding the denatured RNA of the composition produced in (c) to an oligo-dT resin at a temperature of lower than 40° C.; and (e) eluting RNA from the oligo-dT resin.

In Vitro Transcription (IVT) Reaction Mixture

In some embodiments, the mixture or RNA composition to be purified or isolated using the methods described herein is produced by an in vitro transcription (IVT) reaction. In some embodiments, IVT reactions require a linear DNA template containing a promoter, nucleoside triphosphates, a buffer system that includes dithiothreitol (DTT) and magnesium ions, and an RNA polymerase. The exact conditions used in the transcription reaction depend on the amount of RNA needed for a specific application. IVT reactions may be performed by incubating a DNA template with an RNA polymerase and nucleoside triphosphates, including GTP, ATP, CTP, and UTP (or nucleotide analogs) in a transcription buffer. In some embodiments, an RNA polymerase for use in an IVT reaction is as described in WO2019/036682, entitled “RNA Polymerase Variants”. In some embodiments, an IVT reaction is a bolus fed-batch IVT reaction, a continuous fed-batch IVT reaction, or a batch IVT reaction. In some embodiments, an RNA transcript having a 5′ cap structure is produced from this reaction.

A DNA template may include a polynucleotide encoding a polypeptide of interest (e.g., an antigenic polypeptide). A DNA template, in some embodiments, includes an RNA polymerase promoter (e.g., a T7 RNA polymerase promoter) that is operably linked to a polynucleotide encoding a polypeptide of interest. In some embodiments, a DNA template can be transcribed by an RNA polymerase. A DNA template may also include a nucleotide sequence encoding a poly-Adenylation (poly-A) tail at the 3′ end of the polynucleotide encoding a polypeptide of interest.

An RNA, in some embodiments, is the product of an IVT reaction. An RNA, in some embodiments, is a messenger RNA (mRNA) that includes a nucleotide sequence encoding a polypeptide of interest linked to a poly-A tail.

A “poly-A tail” is a region of RNA that contains multiple, consecutive adenosine monophosphates that is downstream, from the region encoding a polypeptide of interest (e.g., directly downstream of the 3′ untranslated region). A poly-A tail may contain 10 to 300 adenosine monophosphates. For example, a poly-A tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a poly-A tail contains 50 to 250 adenosine monophosphates. In some embodiments, a poly-A tail may contain fewer than 10 adenosine monophosphates (e.g., 2, 3, 4, 5, 6, 7, 8, or 9).

In some embodiments, percent tailed RNA (the percent of RNA transcripts comprising a poly-A tail) is greater than 50%, greater than 60%, greater than 70%, greater than 80%, greater than 90%, greater than 95% following an IVT reaction. As used herein, percent tailed RNA generally refers to the relative abundance of transcribed RNA product that contains a 3′ poly-A tail. In some embodiments, percent tailed RNA (the percent of transcribed RNA product comprising a 3′ poly-A tail) is greater than 20%, 25%, 30%, 35%, 40%, 45%, 50%, 55%, 60%, 65%, 70%, 75%, 80%, or 85%. In some embodiments, percent tailed RNA is greater than greater than 90%, 95%, 97%, or 99%. In some embodiments, percent tailed RNA (the percent of transcribed RNA product comprising a 3′ poly-A tail) is 20-100%, 20-90%, 20-80%, 20-70%, 20-60%, 20-50%, 20-40%, 20-30%, 25-75%, 30-50%, 40-60%, 50-70%, 45-60%, 55-70%, 60-80%, 60-100%, 75-100%, 50-95%, 75-95%, 80-100%, 80-90%, 90-95%, 95-100%, 90-99%, or 95-99%.

In some embodiments, the RNA is not chemically modified and comprises the standard ribonucleotides consisting of adenosine, guanosine, cytosine and uridine. In some embodiments, nucleotides and nucleosides of the present disclosure comprise standard nucleoside residues (e.g. A, G, C, or U). In some embodiments, nucleotides and nucleosides of the present disclosure comprise standard deoxyribonucleosides such as those present in DNA (e.g. dA, dG, dC, or dT).

In some embodiments, the RNA is a modified mRNA (mmRNA) and includes at least one modified nucleotide. In some embodiments, the terms “modification” and “modified” refers to modification with respect to adenosine (A), guanosine (G), uridine (U), thymidine (T) or cytidine (C) ribonucleosides or deoxyribonucleosides in at least one of their nucleobase positions, pattern, percent or population. The RNA may comprise modifications that are naturally-occurring, non-naturally-occurring or the RNA may comprise a combination of naturally-occurring and non-naturally-occurring modifications. The RNA may include any useful modification, for example, of a sugar, a nucleobase, or an internucleoside linkage (e.g., to a linking phosphate, to a phosphodiester linkage or to the phosphodiester backbone).

The RNA, in some embodiments, comprises modified nucleosides and/or nucleotides. A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside, including a phosphate group. In some embodiments, modified nucleobases in RNA are selected from the group consisting of pseudouridine (ψ), N1-methylpseudouridine (m1ψ), N1-ethylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine. In some embodiments, an RNA includes a combination of at least two (e.g., 2, 3, 4 or more) of the aforementioned modified nucleobases.

In some embodiments, modified nucleobases in RNA are selected from the group consisting of 1-methyl-pseudouridine (m1ψ), 5-methoxy-uridine (mo5U), 5-methyl-cytidine (m5C), pseudouridine (ψ), α-thio-guanosine and α-thio-adenosine. In some embodiments, an RNA includes a combination of at least two (e.g., 2, 3, 4 or more) of the aforementioned modified nucleobases.

In some embodiments, an RNA comprises pseudouridine (ψ) and 5-methyl-cytidine (m5C). In some embodiments, an RNA comprises 1-methyl-pseudouridine (m1ψ). In some embodiments, an RNA comprises 1-methyl-pseudouridine (m1ψ) and 5-methyl-cytidine (m5C). In some embodiments, an RNA comprises 2-thiouridine (s2U). In some embodiments, an RNA comprises 2-thiouridine and 5-methyl-cytidine (m5C). In some embodiments, an RNA comprises methoxy-uridine (mo5U). In some embodiments, an RNA comprises 5-methoxy-uridine (mo5U) and 5-methyl-cytidine (m5C). In some embodiments, an RNA comprises 2′-O-methyl uridine. In some embodiments an RNA comprises 2′-O-methyl uridine and 5-methyl-cytidine (m5C). In some embodiments, an RNA comprises N6-methyl-adenosine (m6A). In some embodiments, an RNA comprises N6-methyl-adenosine (m6A) and 5-methyl-cytidine (m5C).

The RNA may contain from about 1% to about 100% modified nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of unmodified A, G, U, or C.

In some embodiments, the RNA comprises a cap analog. An RNA cap analog generally enhances mRNA stability and translation efficiency. Traditional cap analogs include GpppG, m7GpppG, and m2,2,7GpppG. In some embodiments, an RNA cap analog of the present disclosure is a dinucleotide cap, a trinucleotide cap, or a tetranucleotide cap. In some embodiments, the cap analog is a trinucleotide cap. In some embodiments, the trinucleotide cap comprises a sequence selected from the following sequences: GAA, GAC, GAG, GAU, GCA, GCC, GCG, GCU, GGA, GGC, GGG, GGU, GUA, GUC, GUG, and GUU.

In some embodiments, the trinucleotide cap comprises a sequence selected from the following sequences: m⁷GpppApA, m⁷GpppApC, m⁷GpppApG, m⁷GpppApU, m⁷GpppCpA, m⁷GpppCpC, m⁷GpppCpG, m⁷GpppCpU, m⁷GpppGpA, m⁷GpppGpC, m⁷GpppGpG, m⁷GpppGpU, m⁷GpppUpA, m⁷GpppUpC, m⁷GpppUpG, and m⁷GpppUpU. In some embodiments, the trinucleotide cap comprises a sequence selected from the following sequences: m⁷G_{3′OMe}pppApA, m⁷G_{3′OMe}pppApC, m⁷G_{3′OMe}pppApG, m⁷G_{3′OMe}pppApU, m⁷G_{3′OMe}pppCpA, m⁷G_{3′OMe}pppCpC, m⁷G_{3′OMe}pppCpG, m⁷G_{3′OMe}pppCpU, m⁷G_{3′OMe}pppGpA, m⁷G_{3′OMe}pppGpC, m⁷G_{3′OMe}pppGpG, m⁷G_{3′OMe}pppGpU, m⁷G_{3′OMe}pppUpA, m⁷G_{3′OMe}pppUpC, m⁷G_{3′OMe}pppUpG, and m⁷G_{3′OMe}pppUpU. In some embodiments, the trinucleotide cap comprises a sequence selected from the following sequences: m⁷G_{3′OMe}pppA_{2′OMe}pA, m⁷G_{3′OMe}pppA_{2′OMe}pC, m⁷G_{3′OMe}pppA_{2′OMe}pG, m⁷G_{3′OMe}pppA_{2′OMe}pU, m⁷G_{3′OMe}pppC_{2′OMe}pA, m⁷G_{3′OMe}pppC_{2′OMe}pC, m⁷G_{3′OMe}pppC_{2′OMe}pG, m⁷G_{3′OMe}pppC_{2′OMe}pU, m⁷G_{3′OMe}pppG_{2′OMe}pA, m⁷G_{3′OMe}pppG_{2′OMe}pC, m⁷G_{3′OMe}pppG_{2′OMe}pG, m⁷G_{3′OMe}pppG_{2′OMe}pU, m⁷G_{3′OMe}pppU_{2′OMe}pA, m⁷G_{3′OMe}pppU_{2′OMe}pC, m⁷G_{3′OMe}pppU_{2′OMe}pG, and m⁷G_{3′OMe}pppU_{2′OMe}pU. In some embodiments, the trinucleotide cap comprises a sequence selected from the following sequences: m⁷GpppA_{2′OMe}pA, m⁷GpppA_{2′OMe}pC, m⁷GpppA_{2′OMe}pG, m⁷GpppA_{2′OMe}pU, m⁷GpppC_{2′OMe}pA, m⁷GpppC_{2′OMe}pC, m⁷GpppC_{2′OMe}pG, m⁷GpppC_{2′OMe}pU, m⁷GpppG_{2′OMe}pA, m⁷GpppG_{2′OMe}pC, m⁷GpppG_{2′OMe}pG, m⁷GpppG_{2′OMe}pU, m⁷GpppU_{2′OMe}pA, m⁷GpppU_{2′OMe}pC, m⁷GpppU_{2′OMe}pG, and m⁷GpppU_{2′OMe}pU.

As used herein, percent capped RNA generally refers to the relative abundance of transcribed RNA product that contains an incorporated cap analog at its 5′ terminus. In some embodiments, a cap analog is an RNA cap analog. In some embodiments, an RNA cap analog is a dinucleotide, trinucleotide, or tetranucleotide. In some embodiments, percent capped RNA (the percent of transcribed RNA product comprising a 5′ cap analog) is greater than 20%, 25%, 30%, 35%, 40%, 45%, 50%, 55%, 60%, 65%, 70%, 75%, 80%, or 85%. In some embodiments, percent capped RNA is greater than greater than 90%, 95%, 97%, or 99%. In some embodiments, percent capped RNA is between 20-100%, 20-90%, 20-80%, 20-70%, 20-60%, 20-50%, 20-40%, 20-30%, 25-75%, 30-50%, 40-60%, 50-70%, 45-60%, 55-70%, 60-80%, 60-100%, 75-100%, 50-95%, 75-95%, 80-100%, 80-90%, 90-95%, 95-100%, 90-99%, or 95-99%.

The RNA may be any size or length. In some embodiments, the RNA is 50-250, 200-500, 400-5000, 400-4000, 400-3000, 400-2000, 400-1000, 500-5000, 500-1500, 750-2000, 1000-1500, 1250-2000, 1500-2000, 1750-2500, 2000-3000, 2500-3500, 3000-4000, 3500-4500, or 4000-5000 nucleotides in length.

Desalting Mixtures Comprising RNA

In some embodiments, mixtures comprising RNA are desalted in order to produce low-salt RNA compositions (e.g., having less than 20 mM total salt concentration). In some embodiments, a mixture comprising RNA (e.g., a mixture produced by an IVT reaction) is desalted prior to denaturation of the RNA and/or in-line mixing with a high-salt buffer.

In some embodiments, a low-salt RNA composition comprises sodium, potassium, magnesium, manganese, calcium, sulfate, phosphate, and/or chloride salts. In some embodiments, a low-salt RNA composition comprises sodium chloride, sodium phosphate, sodium sulfate, potassium chloride, potassium phosphate, potassium sulfate, magnesium chloride, magnesium phosphate, magnesium sulfate, calcium chloride, calcium phosphate, and/or calcium sulfate. In some embodiments, a low-salt RNA composition comprises a total salt concentration of less than 20 mM, less than 15 mM, less than 10 mM, less than 5 mM, or less than 1 mM. In some embodiments, a low-salt RNA composition comprises a salt concentration of 1-20 mM, 1-15 mM, 1-10 mM, 1-5 mM, 5-20 mM, 5-10 mM, 10-20 mM, 10-15 mM or 15-20 mM. In some embodiments, a low-salt RNA composition results in a conductivity of less than 2.5 mS/cm, less than 2 mS/cm, less than 1.5 mS/cm, less than 1 mS/cm, or less than 0.5 mS/cm. In some embodiments, a low-salt RNA composition comprises a conductivity of 0.1-2.5 mS/cm, 0.1-2 mS/cm, 0.5-2 mS/cm, 0.5-1 mS/cm, 1-2 mS/cm, 1-1.5 mS/cm, or 1-1.25 mS/cm.

In some embodiments, desalting a mixture comprising RNA is accomplished by binding the RNA to a hydrophobic interaction chromatography (HIC) resin and eluting the RNA from the HIC resin to produce the low-salt RNA composition. In some embodiments, the HIC resin is equilibrated with a buffer prior to binding the RNA to the resin. In some embodiments, the HIC resin is equilibrated with a buffer comprising 100 mM NaCl, 10 mM Tris, 1 mM EDTA pH 7.4. In some embodiments, the RNA is eluted from the HIC resin using water or a buffer.

The methods described here may comprise any HIC resin. In some embodiments, the HIC resin comprises butyl, t-butyl, methyl, and/or ethyl functional groups. In some embodiments, the HIC resin is a HiTrap Butyl HP resin, CaptoPhenyl resin, Phenyl Sepharose™ 6 resin, Phenyl Sepharose™ High Performance resin, Octyl Sepharose™ High Performance resin, Fractogel™ EMD Propyl resin, Fractogel™ EMD Phenyl resin, Macro-Prep™ Methyl resin, HiScreen Butyl FF, HiScreen Octyl FF, or Tosoh Hexyl. In some embodiments, the HIC resin is a (poly)styrene-divinylbenzene (PS-DVB) R150 bead resin with 2000 Angstrom pores.

In some embodiments, desalting a mixture comprising RNA is accomplished by dilution of the mixture with water (e.g., a 10× water dilution), tangential flow filtration (TFF) of the mixture into water, or ambient oligo-dT (i.e., under native, non-denaturing RNA conditions).

Denatured RNA

In some embodiments, an RNA composition (e.g., a low-salt RNA composition) is denatured. In some embodiments, a low-salt RNA composition is denatured prior to (e.g., immediately prior to) in-line mixing with a high-salt buffer and subsequent binding of the denatured RNA to an oligo-dT resin.

RNA may be denatured using any method. In some embodiments, RNA is denatured by heating the low-salt RNA composition to 60° C. to 90° C., 60° C. to 80° C., 60° C. to 70° C., 65° C. to 85° C., 65° C. to 75° C., 70° C. to 90° C., 70° C. to 80° C., 65° C. to 70° C., 70° C. to 75° C., or 75° C. to 95° C. In some embodiments, the low-salt RNA composition is heated for less than 2 minutes, less than 90 seconds, less than 1 minute, less than 50 seconds, less than 40 seconds, less than 30 seconds, less than 20 seconds, or less than 10 seconds. In some embodiments, the low-salt RNA composition is heated for 5-90 seconds, 5-60 seconds, 5-30 seconds, 5-15 seconds, 10-60 seconds, 10-30 seconds, 20-60 seconds, 20-40 seconds, 30-90 seconds, 30-60 seconds, 40-90 seconds, 40-60 seconds, 60-120 seconds, 60-90 seconds, or 90-120 seconds. In some embodiments, the high-salt RNA composition is heated in the presence of a denaturant molecule (e.g., a chemical small molecule that destabilizes or denatures RNA). A denaturant molecule may include dimethyl sulfoxide (e.g., at a concentration of 0.05-1% v/v, 0.1-0.5% v/v, 0.05-0.5% v/v, or 0.25-0.75% v/v), guanidine (e.g., at a concentration of 50-250 mM, 100-500 mM, 250-1000 mM, 1-8 M, 2-6 M, 3-5 M, or 5-8 M), or urea (e.g., at a concentration of 50-250 mM, 100-500 mM, 250-1000 mM, 1-8 M, 2-6 M, 3-5 M, or 5-8 M).

In some embodiments, a change in the relative amount of denatured RNA in an RNA composition during a denaturation process (e.g., heating the low-salt RNA composition to 60° C. to 90° C., 60° C. to 80° C., 60° C. to 70° C., 65° C. to 85° C., 65° C. to 75° C., 70° C. to 90° C., 70° C. to 80° C., 65° C. to 70° C., 70° C. to 75° C., or 75° C. to 95° C.) is determined by hyperchromicity curves (e.g., spectroscopic melting curves). In some embodiments, a change in the relative amount of denatured RNA is determined by measuring the change in secondary structure of the total RNA in a composition (e.g., by determining a change in ultraviolet absorption). In some embodiments, a change in the relative amount of denatured RNA is determined by monitoring the change in secondary structure of the total RNA in a composition (e.g., by determining a change in ultraviolet absorption) before and after the denaturation process (e.g., heating the low-salt RNA composition to 60° C. to 90° C., 60° C. to 80° C., 60° C. to 70° C., 65° C. to 85° C., 65° C. to 75° C., 70° C. to 90° C., 70° C. to 80° C., 65° C. to 70° C., 70° C. to 75° C., or 75° C. to 95° C.).

In some embodiments, at least 50%, 55%, 60%, 65%, 70%, 75%, 80%, or 85% of the total RNA in a denatured RNA composition comprises denatured RNA. In some embodiments, at least 90% (e.g., at least 91%, 92%, 93%, 94%, 95%, 96%, 97%, 98%, 99% or 100%) of the total RNA in a denatured RNA composition comprises denatured RNA. In some embodiments, 50-70%, 45-60%, 55-70%, 60-80%, 60-100%, 75-100%, 50-95%, 75-95%, 80-100%, 80-90%, 90-95%, 95-100%, 90-99%, or 95-99% of the total RNA in a denatured RNA composition comprises denatured RNA. In some embodiments, the relative amount of denatured RNA in a denatured RNA composition is determined by hyperchromicity curves (e.g., spectroscopic melting curves). Hyperchromicity, the property of nucleic acids such as RNA to exhibit an increase in extinction coefficient upon the loss of structure during heating, may be measured (e.g., during denaturation of RNA, e.g., by heating) using a spectrophotometer. In some embodiments, the extinction coefficient of RNA is measured at 205 nm, 220 nm, 260 nm, or 200-300 nm. In some embodiments, the relative amount of denatured RNA in a denatured RNA composition is determined using a method as described in S. J. Schroeder and D. H. Turner, “Optical melting measurements of nucleic acid thermodynamics”, Methods Enzymol. 468 (2009) 371-387; or Gruenwedel, D. W., “Nucleic Acids: Properties and Determination”, Encyclopedia of Food Sciences and Nutrition, 2003, Pages 4147-4152.

In some embodiments, a denatured RNA composition is stored in a break tank (i.e., a storage container that can hold the denatured RNA composition) prior to mixing with a high-salt buffer and/or loading onto an oligo-dT resin. In some embodiments, the break tank is capable of storing the denatured RNA composition such that the RNA remains denatured for up to 3 days. In some embodiments, the denatured RNA composition is maintained at a low salt concentration (e.g., 1-20 mM, 1-15 mM, 1-10 mM, 1-5 mM, 5-20 mM, 5-10 mM, 10-20 mM, 10-15 mM or 15-20 mM salt) in the break tank. In some embodiments, the break tank is capable of storing the denatured RNA composition such that the RNA remains denatured for 1-6 hours, 2-12 hours, 5-15 hours, 12-24 hours, 12-36 hours, 1-2 days, 1-3 days, or 2-3 days. In some embodiments, the break tank is maintained at 15° C. to 30° C., 4° C. to 30° C., 4° C. to 25° C., 4° C. to 20° C., 4° C. to 15° C., 4° C. to 10° C., 4° C. to 8° C., 10° C. to 30° C., 10° C. to 25° C., 10° C. to 20° C., 10° C. to 15° C. or 15° C. to 25° C.

In-Line Mixing

In some embodiments, in-line mixing refers to mixing of a first continuous stream of a solution with a second continuous stream of a solution. In some embodiments, the first and second continuous streams are controlled by independent pumps (e.g., independent peristaltic pumps). In some embodiments, in-line mixing relies on flow control conditions, for example, process flow conditions wherein flow parameters (e.g., flow rate, temperature) are controlled by a flow regulating device comprising at least one pump system. In some embodiments, the first continuous stream is a high-salt buffer (e.g., comprising at least 50 mM salt), and the second continuous stream is a composition comprising desalted (e.g., low-salt) and/or denatured RNA.

In-line mixing typically occurs shortly prior to binding a composition comprising denatured RNA to an oligo-dT resin. In some embodiments, in-line mixing occurs for less than 2 minutes, less than 90 seconds, less than 1 minute, less than 50 seconds, less than 40 seconds, less than 30 seconds, less than 20 seconds, or less than 10 seconds. In some embodiments, in-line mixing occurs for 5-90 seconds, 5-60 seconds, 5-30 seconds, 5-15 seconds, 10-60 seconds, 10-30 seconds, 20-60 seconds, 20-40 seconds, 30-90 seconds, 30-60 seconds, 40-90 seconds, 40-60 seconds, 60-120 seconds, 60-90 seconds, or 90-120 seconds. In some embodiments, in-line mixing occurs 5-90 seconds, 5-60 seconds, 5-30 seconds, 5-15 seconds, 10-60 seconds, 10-30 seconds, 20-60 seconds, 20-40 seconds, 30-90 seconds, 30-60 seconds, 40-90 seconds, 40-60 seconds, 60-120 seconds, 60-90 seconds, or 90-120 seconds prior to binding a composition comprising denatured RNA to an oligo-dT resin. In some embodiments, in-line mixing occurs at a temperature of 4° C. to 30° C., 4° C. to 25° C., 4° C. to 20° C., 4° C. to 15° C., 4° C. to 10° C., 4° C. to 8° C., 10° C. to 30° C., 10° C. to 25° C., 10° C. to 20° C., 10° C. to 15° C., or 15° C. to 25° C.

In some embodiments, a high-salt buffer (e.g., that may be in-line mixed with an RNA composition) comprises a salt concentration of at least 50 mM, at least 60 mM, at least 70 mM, at least 80 mM, at least 90 mM, at least 100 mM, at least 125 mM, at least 150 mM, at least 200 mM, at least 250 mM, at least 300 mM, at least 350 mM, at least 400 mM, at least 500 mM, at least 600 mM, at least 700 mM, at least 800 mM, at least 900 mM, or at least 1000 mM. In some embodiments, a high-salt buffer comprises a salt concentration of 50-500 mM, 50-250 mM, 50-100 mM, 50-75 mM, 60-150 mM, 75-500 mM, 75-200 mM, 100-500 mM, 100-250 mM, 150-350 mM, 200-400 mM, 250-500 mM, 300-400 mM, 350-450 mM, 400-500 mM, 400-600 mM, 500-700 mM, 500-750 mM, 700-1000 mM, 750-900 mM, or 850-1000 mM. In some embodiments, a high-salt buffer comprises a salt concentration of 1-2 M, 2-3 M, 3-4 M, or 4-5 M. In some embodiments, a high-salt buffer comprises a conductivity of at least 5 mS/cm, at least 6 mS/cm, at least 7 mS/cm, at least 8 mS/cm, at least 9 mS/cm, at least 10 mS/cm, at least 12 mS/cm, or at least 15 mS/cm. In some embodiments, a high-salt buffer comprises a conductivity of 5-10 mS/cm, 5-15 mS/cm, 5-7 mS/cm, 6-9 mS/cm, 8-10 mS/cm, 9-12 mS/cm, or 10-15 mS/cm.

In some embodiments, in-line mixing a composition comprising denatured RNA with a high-salt buffer produces a composition comprising denatured RNA and salt at a concentration of at least 50 mM. In some embodiments, in-line mixing a composition comprising denatured RNA with a high-salt buffer produces a composition comprising denatured RNA and salt at a concentration of 50-500 mM, 50-250 mM, 50-100 mM, 50-75 mM, 60-150 mM, 75-500 mM, 75-200 mM, 100-500 mM, 100-250 mM, 150-350 mM, 200-400 mM, 250-500 mM, 300-400 mM, 350-450 mM, 400-500 mM, 400-600 mM, 500-700 mM, 500-750 mM, 700-1000 mM, 750-900 mM, or 850-1000 mM. In some embodiments, in-line mixing a composition comprising denatured RNA with a high-salt buffer produces a composition comprising denatured RNA and salt having a conductivity of less than 2 mS/cm. In some embodiments, in-line mixing a composition comprising denatured RNA with a high-salt buffer produces a composition comprising denatured RNA and salt having a conductivity of 2-5 mS/cm, 2-7 mS/cm, 5-10 mS/cm, 5-15 mS/cm, 5-7 mS/cm, 6-9 mS/cm, 8-10 mS/cm, 9-12 mS/cm, or 10-15 mS/cm.

In some embodiments, a buffer (e.g., a low-salt or high-salt buffer) comprises NaCl, KCl, LiCl, NaH₂PO₄, Na₂HPO₄, or Na₃PO₄. In some embodiments, a buffer (e.g., a low-salt or high-salt buffer) comprises any source of sodium, potassium, magnesium, phosphate, chloride, or any other source of salt ions. In some embodiments, a buffer (e.g., a low-salt or high-salt buffer) may further comprise a buffering agent in order to maintain a consistent pH. In some embodiments, a buffer (e.g., a low-salt or high-salt buffer) comprises a neutral pH. In some embodiments, a buffer (e.g., a low-salt or high-salt buffer) comprises a pH of about 6, about 6.5, about 7, about 7.4, about 8, or about 6-8. Examples of buffering agents for use herein include ethylenediamine tetraacetic acid (EDTA), succinate, citrate, aspartic acid, glutamic acid, maleate, cacodylate, 2-(N-morpholino)-ethanesulfonic acid (MES), N-(2-acetamido)-2-aminoethanesulfonic acid (ACES), piperazine-N,N′-2-ethanesulfonic acid (PIPES), 2-(N-morpholino)-2-hydroxypropanesulfonic acid (MOPSO), N,N-bis-(hydroxyethyl)-2-aminoethanesulfonic acid (BES), 3-(N-morpholino)-propanesulfonic acid (MOPS), N-2-hydroxyethyl-piperazine-N-2-ethanesulfonic acid (HEPES), 3-(N-tris-(hydroxymethyl)methylamino)-2-hydroxypropanesulfonic acid (TAPSO), 3-(N,N-bis[2-hydroxyethyl]amino)-2-hydroxypropanesulfonic acid (DIPSO), N-(2-hydroxyethyl)piperazine-N′-(2-hydroxypropanesulfonic acid) (HEPPSO), 4-(2-hydroxyethyl)-1-piperazine propanesulfonic acid (EPPS), N-[tris(hydroxymethyl)-methyl]glycine (Tricine), N,N-bis(2-hydroxyethyl)glycine (Bicine), [(2-hydroxy-1,1-bis(hydroxymethyl)ethyl)amino]-1-propanesulfonic acid (TAPS), N-(1,1-dimethyl-2-hydroxyethyl)-3-amino-2-hydroxypropanesulfonic acid (AMPSO), tris(hydroxymethyl)aminomethane (Tris), and bis[2-hydroxyethyl]iminotris-[hydroxymethyl]methane (Bis-Tris). Other buffers compositions, buffer concentrations, and additional components of a solution for use herein will be apparent to those skilled in the art.

In some embodiments, in-line mixing comprises in-line cooling of a composition comprising denatured RNA to a temperature of 4° C. to 30° C., 4° C. to 25° C., 4° C. to 20° C., 4° C. to 15° C., 4° C. to 10° C., 4° C. to 8° C., 10° C. to 30° C., 10° C. to 25° C., 10° C. to 20° C., 10° C. to 15° C., or 15° C. to 25° C. In some embodiments, in-line mixing comprises in-line cooling of a composition comprising denatured RNA to a temperature below 60° C., 55° C., 50° C., 45° C., 40° C., 35° C., 30° C., 25° C., 20° C., 15° C., 10° C., or 5° C. In some embodiments, in-line cooling occurs simultaneously with in-line mixing of a composition comprising denatured RNA and low salt buffer with a high salt buffer. In some embodiments, during in-line cooling, a composition comprising denatured RNA is maintained at a total salt concentration of less than 20 mM, less than 15 mM, less than 10 mM, less than 5 mM, or less than 1 mM. In some embodiments, during in-line cooling, a composition comprising denatured RNA is maintained at a total salt concentration of 1-20 mM, 1-15 mM, 1-10 mM, 1-5 mM, 5-20 mM, 5-10 mM, 10-20 mM, 10-15 mM or 15-20 mM. In some embodiments, in-line cooling occurs simultaneously with in-line mixing of a composition comprising denatured RNA and low salt buffer with a high salt buffer. In some embodiments, during in-line cooling, a composition comprising denatured RNA is maintained at less than 2.5 mS/cm, less than 2 mS/cm, less than 1.5 mS/cm, less than 1 mS/cm, or less than 0.5 mS/cm. In some embodiments, in-line cooling occurs simultaneously with in-line mixing of a composition comprising denatured RNA and low salt buffer with a high salt buffer. In some embodiments, during in-line cooling, a composition comprising denatured RNA is maintained at 0.1-2.5 mS/cm, 0.1-2 mS/cm, 0.5-2 mS/cm, 0.5-1 mS/cm, 1-2 mS/cm, 1-1.5 mS/cm, or 1-1.25 mS/cm.

Oligo-dT Resin

The methods herein involve binding (i.e., contacting) compositions comprising denatured RNA to oligo-dT resin. In some embodiments, methods herein comprise binding compositions comprising denatured RNA to oligo-dT resin following in-line mixing of low-salt denatured RNA composition with high-salt buffers.

The methods described herein may use any oligo-dT resin. In some embodiments, the oligo-dT resin is a (poly)styrene-divinylbenzene (PS-DVB) bead resin with 2000 Angstrom pores derivatized with poly dT. In some embodiments, poly dT comprises 5-200, 10-50, 10-100, 50-200, 100-150, or 125-200 thymidines and/or uracils. In some embodiments, poly dT comprises 20 thymidines in length. In some embodiments, poly dT is linked directly to the bead resin. In some embodiments, poly dT is linked to the bead resin via a linker.

In some embodiments, the oligo-dT resin is equilibrated with a buffer prior to binding the RNA to the resin. In some embodiments, the oligo-dT resin is equilibrated with a buffer comprising 100 mM NaCl, 10 mM Tris, and 1 mM EDTA at pH 7.4. In some embodiments, the oligo-dT resin is washed with a buffer after the RNA is bound to the resin. In some embodiments, the washing step comprises a buffer comprising 100 mM NaCl, 10 mM Tris, and 1 mM EDTA, at pH 7.4.

In some embodiments, the binding of the RNA to the oligo-dT resin occurs at a temperature of lower than 40° C. In some embodiments, the binding of the RNA to the oligo-dT resin occurs at a temperature of 4° C. to 30° C., 4° C. to 25° C., 4° C. to 20° C., 4° C. to 15° C., 4° C. to 10° C., 4° C. to 8° C., 10° C. to 30° C., 10° C. to 25° C., 10° C. to 20° C., 10° C. to 15° C., or 15° C. to 25° C.

In some embodiments, the composition comprising denatured RNA is bound to or in contact with the oligo-dT resin for a total residence time of less than 20 minutes, less than 18 minutes, less than 15 minutes, less than 12 minutes, less than 10 minutes, less than 9 minutes, less than 8 minutes, less than 7 minutes, less than 6 minutes, less than 5 minutes, less than 4 minutes, less than 3 minutes, less than 2 minutes, or less than 1 minute. In some embodiments, the composition comprising denatured RNA is bound to or in contact with the oligo-dT resin for a total residence time of 1-2, 1-5, 2-5, 2-10, 5-20, 5-10, 5-15, 8-15, 10-15, 12-20, or 15-20 minutes.

In some embodiments, the methods comprise eluting RNA from the oligo-dT resin. In some embodiments, the RNA is eluted from the HIC resin using water or a buffer (e.g., a buffer comprising 10 mM Tris, 1 mM EDTA, at pH 8.0).

In some embodiments, the RNA eluted from the oligo-dT resin comprises at least 50%, 55%, 60%, 65%, 70%, 75%, 80%, or 85% poly-A tailed RNA. In some embodiments, the RNA eluted from the oligo-dT resin comprises about 20-100%, 20-90%, 20-80%, 20-70%, 20-60%, 20-50%, 20-40%, 20-30%, 25-75%, 30-50%, 40-60%, 50-70%, 45-60%, 55-70%, 60-80%, 60-100%, 75-100%, 50-95%, 75-95%, 80-100%, 80-90%, 90-95%, 95-100%, 90-99%, or 95-99% poly-A tailed mRNA. In some embodiments, the RNA eluted from the oligo-dT resin comprises at least 91%, 92%, 93%, 94%, 95%, 96%, 97%, 98%, 99% or 100% poly-A tailed mRNA.

Apparatus

Some aspects of the present disclosure provide an apparatus for purifying RNA using denaturing dT chromatography. In some embodiments, the apparatus comprises a column packed with oligo-dT resin, the column having an inlet and an outlet. In some embodiments, the apparatus is a flow regulating device comprising at least one pump system, wherein the pump system allows for continuous blend mixing of two or more solutions under flow control conditions (e.g., process flow conditions) to control flow parameters such as flow rate and temperature of the two or more solutions to be mixed. In some embodiments, the apparatus comprises, upstream of the inlet of the column, a first continuous stream for delivering desalted RNA, wherein the flow of desalted RNA is controlled by a first pump, and wherein the first stream in encased within a denaturation chamber comprising a pre-heater followed by a chiller; a second stream for delivering high-salt buffer, wherein the flow of high-salt buffer is controlled by a second pump; and a chamber where the two continuous streams are combined to provide in-line mixing of the desalted RNA and high-salt buffer.

In some embodiments, the apparatus comprises an oligo-dT resin that comprises (poly)styrene-divinylbenzene (PS-DVB) bead resin with 2000 Angstrom pores derivatized with poly dT.

In some embodiments, a column (e.g., a column packed with oligo-dT resin) has a column volume of 1-10 mL, 1-5 mL, 5-25 mL, 10-100 mL, 25-150 mL, 50-100 mL, 75-150 mL, 100-200 mL, 100-500 mL, 250-1000 mL, 500-1500 mL, or more. In some embodiments, a column (e.g., a column packed with oligo-dT resin) has a column volume of about 1 mL, about 5 mL, about 10 mL, about 25 mL, about 50 mL, about 100 mL, about 250 mL, about 500 mL, about 750 mL, about 1000 mL, about 1500 mL, about 2000 mL, or more.

In some embodiments, the pre-heater heats the desalted RNA to a temperature of higher than 60° C. to produce a denatured RNA composition. In some embodiments, the pre-heater is maintained at 60° C. to 90° C., 60° C. to 80° C., 60° C. to 70° C., 65° C. to 85° C., 65° C. to 75° C., 70° C. to 90° C., 70° C. to 80° C., 65° C. to 70° C., 70° C. to 75° C., or 75° C. to 95° C.

In some embodiments, the chiller cools the denatured RNA composition to a temperature of less than 30° C. In some embodiments, the chiller is maintained at 15° C. to 30° C., 4° C. to 30° C., 4° C. to 25° C., 4° C. to 20° C., 4° C. to 15° C., 4° C. to 10° C., 4° C. to 8° C., 10° C. to 30° C., 10° C. to 25° C., 10° C. to 20° C., 10° C. to 15° C., or 15° C. to 25° C.

In some embodiments, the apparatus further comprises a break tank (i.e., a storage container that can hold the denatured RNA composition). In some embodiments, the break tank is capable of storing the denatured RNA composition such that the RNA remains denatured for up to 3 days. In some embodiments, the denatured RNA composition is maintained at a low salt concentration (e.g., 1-20 mM, 1-15 mM, 1-10 mM, 1-5 mM, 5-20 mM, 5-10 mM, 10-20 mM, 10-15 mM or 15-20 mM salt) in the break tank. In some embodiments, the break tank is capable of storing the denatured RNA composition such that the RNA remains denatured for 1-6 hours, 2-12 hours, 5-15 hours, 12-24 hours, 12-36 hours, 1-2 days, 1-3 days, or 2-3 days. In some embodiments, the break tank is maintained at 15° C. to 30° C., 4° C. to 30° C., 4° C. to 25° C., 4° C. to 20° C., 4° C. to 15° C., 4° C. to 10° C., 4° C. to 8° C., 10° C. to 30° C., 10° C. to 25° C., 10° C. to 20° C., 10° C. to 15° C., or 15° C. to 25° C.

In some embodiments, the apparatus further comprises at least one ultraviolet detection (UV) module. In some embodiments, the UV detection module is positioned to detect RNA using UV light during the denaturation process (e.g., during a heating step intended to denature the RNA). In some embodiments, the UV detection module is positioned to detect RNA using UV light after the denaturation process (e.g., after a heating step intended to denature the RNA). In some embodiments, the apparatus comprises two UV modules. In some embodiments, the apparatus comprises a first UV module positioned to detect RNA using UV light before the denaturation process (e.g., before a heating step intended to denature the RNA) and a second UV module positioned to detect RNA using UV light after the denaturation process (e.g., after a heating step intended to denature the RNA).

In some embodiments, the apparatus is used to process desalted RNA produced using an in vitro transcription reaction. In some embodiments, the high-salt buffer has a salt concentration of at least 50 mM. In some embodiments, the high-salt buffer has a salt concentration of 50 mM to 500 mM NaCl.

### EXAMPLES

### Example 1. Denaturing dT (ddT) Chromatography

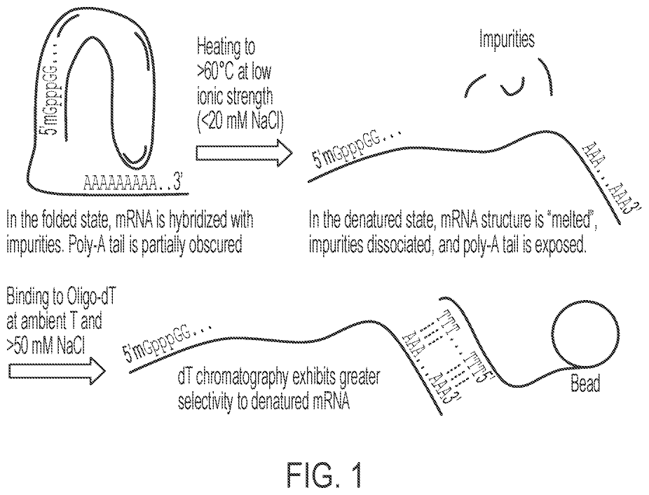

As described in FIG. 1, denaturation improves the selectivity of oligo-dT chromatography for poly-A tail-containing mRNA (e.g., full-length RNA product(s) produced by IVT reactions). Denaturation of RNA compositions can be achieved by heating an RNA solution for 10-60 seconds at temperatures ranging from 60° C. to 90° C. This denaturation process disrupts RNA secondary and higher-order structures, resulting in breaking of any interactions between the mRNA and any non-covalent impurities. Denaturation also causes the dissociation of non-covalently bound IVT-related impurities. The denatured RNA can then be selectively bound to an oligo-dT resin e.g., (poly)styrene-divinylbenzene (PS-DVB) bead resin with 2000 Angstrom pores derivatized with poly dT, and the impurities may be washed away and separated from the denatured RNA.

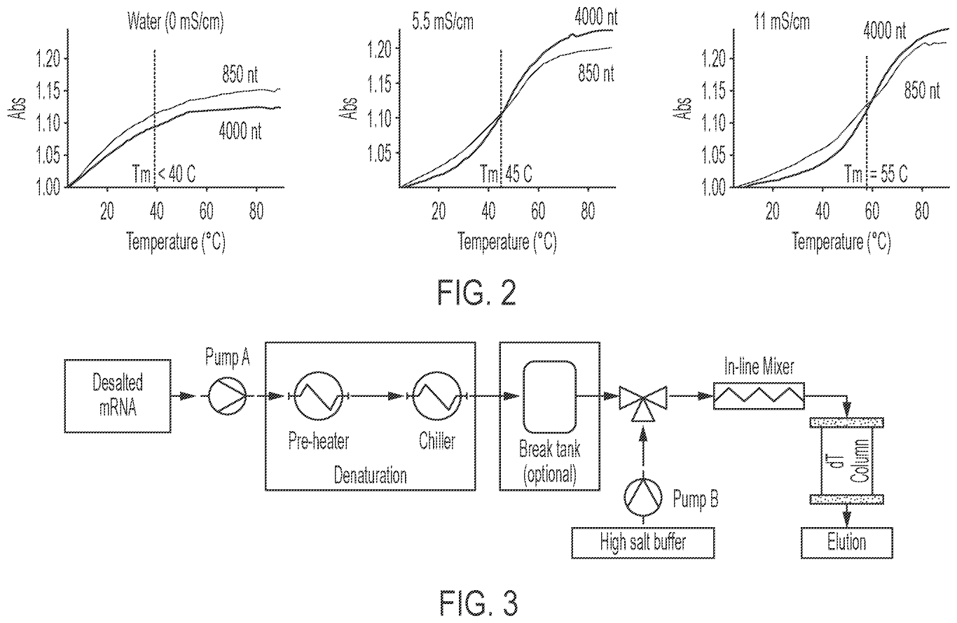

Denaturation of mRNA was studied by examining the effect of salt concentration on the midpoint of melting transition temperature (Tm) (FIG. 2). UV melting curves were obtained using mRNA constructs of two different lengths: 850 nucleotides (nt) and 4000 nt. Constructs were diluted to 0.035 mg/mL in water, 100 mM trimethylamine acetate pH 7.0, and 100 mM sodium acetate pH 7.0. Temperature was ramped from 4° C. to 90° C. at 1° C./minute. Hyperchromicity, the property of nucleic acids to exhibit an increase in extinction coefficient upon the loss of structure during heating, was measured during heating using a Cary-300 spectrophotometer at 260 nm. The midpoint of melting transition was estimated from the resulting hyperchromicity curves and was found to increase with increasing ionic strength of the medium. Thus, the effectiveness of denaturation increases with lower concentration of salt. The melting transition point was not impacted by the length of the mRNA.

Experiments to purify a test mRNA (2525 nt in length) that was prepared using an IVT reaction were then performed to demonstrate that the denaturing oligo-dT methods as described herein provide an optimal RNA purification approach, when compared to alternative purification approaches. A schematic of the apparatus used for the denaturing oligo-dT experiments is shown in FIG. 3.

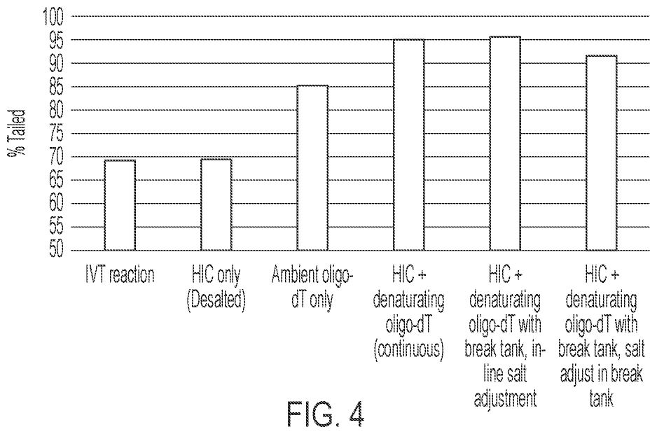

The results of these different approaches are shown in FIG. 4 and described below. Prior to any purification approach, the IVT reaction comprised 69.1% of the total RNA was poly-A tailed mRNA. When the resulting IVT solution was desalted by HIC resin, no change in tailed purity was observed. Ambient oligo-dT resin (i.e., where mRNA is not desalted and denatured, but loaded onto the oligo-dT column in high-salt buffer at ambient temperature)) provided a modest improvement in tailed purity, to 85.3% poly-A tailed mRNA. However, purification using HIC resin (to desalt the mixture) followed by continuous denaturing oligo-dT (including in-line mixing of denatured RNA with high-salt buffer, while loading onto the oligo-dT column) provided a significant improvement in tailed purity, particularly compared to the ambient oligo-dT, up to 94.9% poly-A tailed mRNA. Surprisingly, storage of HIC-desalted and heat-denatured mRNA in the break tank (for 3 days at 2° C.-8° C.) has preserved the denatured character of mRNA, since subsequent in-line mixing with salt buffer during oligo-dT loading similarly provided 95.5% poly-A tailed mRNA. However, salt adjustment during storage of the denatured mRNA in the break tank led to a reduction in tailed purity, down to 91.6%, compared to salt adjustment by in-line mixing, indicating that the impurities slowly associate with full-length mRNA during storage in high salt buffer.

Therefore, it was found that in-line mixing of the denatured RNA with a high-salt buffer immediately before loading onto the oligo-dT resin prevented re-hybridization of the full length product with impurities and resulted in high purity (˜95% of total RNA comprising poly-A tail).

Similar results were seen when the oligo-dT resins were overloaded or under capacity during continuous (i.e., denaturation followed by in-line mixing with a high-salt buffer immediately prior to binding of denatured RNA with oligo-dT resin) denaturing oligo-dT (data not shown).

A further comparison of ambient oligo-dT and denaturing oligo-dT was performed. A test mRNA, 2369 nt in length, was produced using fed-batch IVT. Purity was measured by the percent tailed mRNA. After IVT, the total RNA comprised 64% tailed mRNA. The total RNA was then purified using ambient oligo-dT, resulting in 73% tailed mRNA, before being split into two distinct samples. The first sample was purified a second time using ambient dT to provide 77% tailed mRNA, a minor improvement over the first ambient dT purification step. The second sample was purified using denaturing oligo-dT instead of a second round of ambient dT, and the resulting product was 97% tailed. Further data from this experiment is provided in Table 1.

TABLE 1
Purification of low-purity RNA (2369 nt)

| | Ambient oligo-dT | Denaturing oligo-dT |
|---|---|---|
| Load % tailed | 72.6 | 72.6 |
| Elution % Recovery, Total | 69.6 | 60.6 |
| mRNA | | |
| Elution % Tailed | 76.6 | 96.7 |

### Example 2. Purification Using HIC and Denaturing Oligo-dT

Following in vitro transcription (IVT), the RNA feedstock may be desalted using hydrophobic interaction chromatography (HIC). HIC resin is further capable of removing residual protein and residual undigested DNA.

An IVT reaction that produced a 2525 nt mRNA was incubated with 100 U/mL DNase, subjected to EDTA treatment, and then diluted (4×) prior to loading onto a HIC column. The HIC resin was POROS™ R150, a 2000 Å pore mode (Applied Biosystems). The open pore structure of the bead permits higher binding capacities for large mRNA constructs, for example, those over 2000 nt. The load concentration of RNA in water following IVT was 1-2 mg/mL, and the RNA load challenge target was 5 mg mRNA per mL resin. The residence time/flow rate was 3-5 minutes (150 cm/hr). A summary of the HIC parameters is presented in Table 2.

TABLE 2
HIC Chromatography Parameters

| HIC | Buffer Composition | #CV |
|---|---|---|
| Equilibration | 100 mM NaCl, 10 mM Tris, | 3CV |
| | 1 mM EDTA pH 7.4 | |
| Load | IVT + DNAse + 50 mM EDTA solution | variable |
| Chase | 100 mM NaCl, 10 mM Tris, | 3CV |
| | 1 mM EDTA pH 7.4 | |
| Wash | 60 mM NaCl, 6 mM Tris, | 3CV |
| | 0.6 mM EDTA pH 7.4 | |
| Elution | Water | 3CV |
| Strip | 0.1 M NaOH | 3CV |
| Neutralization | 50 mM Tris pH 7.4 | 3CV |

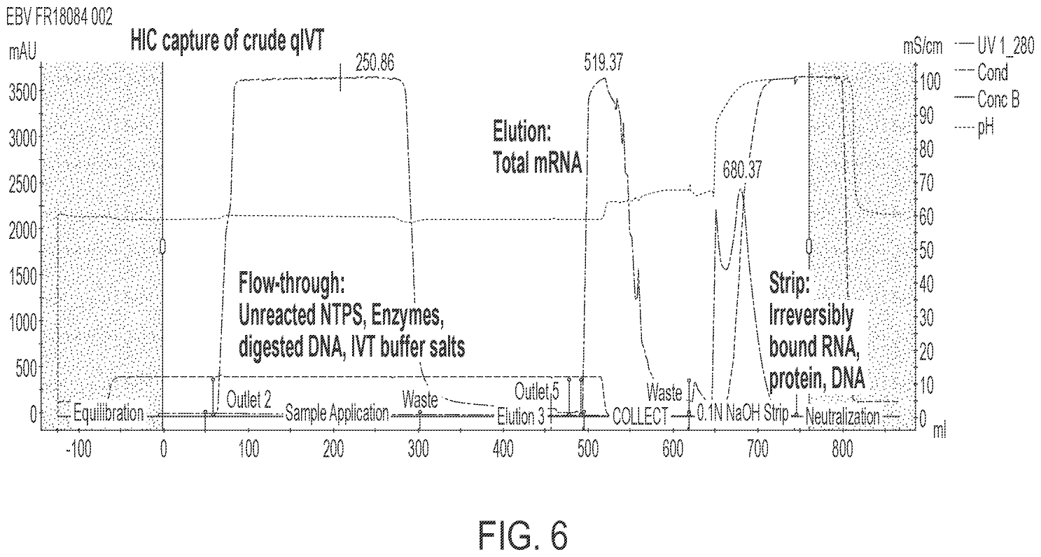

As shown in FIG. 6, HIC permits unreacted NTPs, enzymes, digested DNA, and IVT buffer salts to flow through, while total mRNA can be isolated. Irreversibly bound RNA, protein, and DNA are retained in the column. Elution of the isolated RNA from the HIC resin was then accomplished using water, while undigested DNA was preferentially retained on the column.

The dynamic binding capacity (DBC) of R150 resin from crude IVT feedstock was approximately 5 mg/mL using a 1956 nt mRNA construct and HIC purification provided an unexpected, but modest purity enhancement.

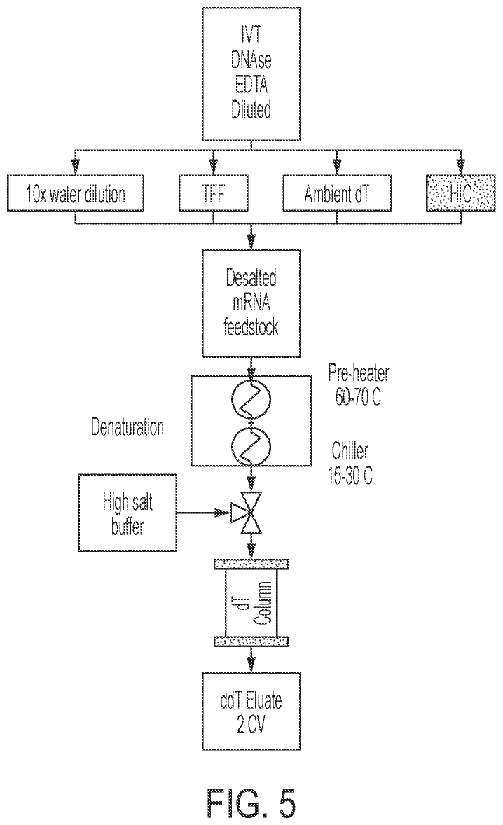

Following the HIC desalting step, the eluted RNA was denatured in-line (See, FIG. 5) by heating the eluted RNA to 60° C. for one minute, chilling to 15-30° C., in-line mixing of the denatured RNA with a buffer comprising 100 mM NaCl, 10 mM Tris, 1 mM EDTA pH 7.4, and loading of the denatured RNA onto an oligo-dT column. The residence time/flow rate was 2 minutes with a 1 mg/mL load. A summary of the HIC parameters is presented in Table 3.

TABLE 3
Oligo-dT Chromatography Parameters

| ddT | Buffer Composition | #CV |
|---|---|---|
| Equilibration | 100 mM NaCl, 10 mM Tris, | 3CV |
| | 1 mM EDTA pH 7.4 | |
| Load | Denatured RNA + (100 mM NaCl, | variable |
| | 10 mM Tris, 1 mM EDTA | |
| | pH 7.4) | |
| Chase | 100 mM NaCl, 10 mM Tris, | 7CV |
| Wash | 1 mM EDTA pH 7.4 | combined |
| Elution | Water | 3CV |
| Strip | 0.1 M NaOH | 3CV |
| Neutralization | 50 mM Tris pH 7.4 | 3CV |

### Example 3. Purification Using HIC and Denaturing Oligo-dT

In this Example, a test mRNA, 1956 nucleotides (nt) in length, was generated using IVT, and then processed using HIC followed by denaturing dT (ddT). As shown in Table 4, the combination of HIC and denaturing oligo-dT processes led to similar or better purity compared to ambient oligo-dT followed by reverse phase (RP) HPLC chromatography. Both processes performed better than dT chromatography alone (center column).

TABLE 4
Analytical Panel: Purification Methods

| | | | HIC + |
|---|---|---|---|
| | Ambient | Ambient | denaturing |
| | oligo-dT + RP | oligo-dT | oligo-dT |
| Purity by FA-CE | 85.0% | 80.0% | 84.5% |
| Purity by HPLC | 84.5% | 77.7% | 83.6% |
| (Size-based) | | | |
| % Poly A tail by | 97.0% | 88.7% | 98.7% |
| HPLC | | | |
| (Tailed/Tailless) | | | |
| Endotoxin (EU/mL) | <0.051 | 0.308 | <0.051 |
| Residual protein by | <LOD | <LOD | <LOD |
| NanoOrange | (<0.29 pg total | (<0.29 pg total | (<0.29 pg total |
| | protein/mL) | protein/mL) | protein/mL) |
| Residual DNA by | 0.62 | 0.69 | 0.29 |
| qPCR, ppm | | | |

The experiment was repeated using mRNA constructs of different sizes: 2992 nt, 2497 nt, 658 nt, 1105 nt, 784 nt, and 913 nt. As shown in Tables 5 and 6 below, a single dT chromatography purification step does not achieve the desired purity for longer constructs (those >2000 nt in length). Using HIC followed by denaturing oligo-dT led to 96-97% tailed mRNA with respect to the longer constructs.

TABLE 5
Percent Tailed mRNA Following Downstream Purification

| | Crude | | | HIC + denaturing | |
|---|---|---|---|---|---|
| Length of | IVT | Ambient oligo-dT | | oligo-dT | |
| construct (nt) | Purity | Purity | Recovery | Purity | Recovery |
| 2992 | 70.6% | 82.7% | 88% | 96.7% | 84% |
| 2497 | 72.4% | 83.4% | 89% | 96.3% | 82% |
| 658 | 92.5% | 96.8% | 96% | 99.1% | 84% |
| 1105 | 89.5% | 94.9% | 94% | 98.8% | 73% |
| 784 | 92.4% | 96.1% | 102% | 99.1% | 82% |
| 913 | 91.1% | 95.9% | 87% | 98.9% | 82% |

TABLE 6
Analytical Panel: Downstream Purification Methods

| | Length | | | | | | |
|---|---|---|---|---|---|---|---|
| | (nt) | 2992 | 2497 | 658 | 1105 | 913 | 784 |
| Ambient | PH | 6.6 | 6.7 | 6.6 | 6.7 | 6.7 | 6.7 |
| oligo-dT | Total RNA | 1.70 | 1.73 | 1.73 | 1.70 | 1.65 | 1.74 |
| | content | | | | | | |
| | (mg/mL) | | | | | | |
| | Purity by | 76.2% | 77.1% | 85.1%3 | 88.8% | 87.9% | 84.9% |
| | FA-CE | | | | | | |
| | Purity by | 84.8% | 81.1% | 88.5%0 | 89.7% | 85.2% | 86.1% |
| | HPLC (Size- | | | | | | |
| | based) | | | | | | |
| | % Poly A | 85.1%) | 85.1% | 97.7% | 93.5% | 96.7% | 97.0% |
| | tail by | | | | | | |
| | HPLC | | | | | | |
| | (Tailed/ | | | | | | |
| | Tailless) | | | | | | |
| | % 5' Cap 1 | 97% | 95% | 89% | 92% | 91% | 90% |
| | by LC/MS | | | | | | |
| | Endotoxin | 0.146 | 0.072 | 0.122 | 0.075 | 0.074 | 0.095 |
| | (EU/mL) | | | | | | |
| | Residual | 1.66 | 2.17 | 2.76 | 20.95 | 15.81 | 1.83 |
| | DNA by | | | | | | |
| | qPCR | | | | | | |
| | (ng/mL) | | | | | | |
| | Residual | <LOD | <LOD | <LOD | <LOD | <LOD | <LOD |
| | protein by | | | | | | |
| | NanoOrange | | | | | | |
| HIC + | Total RNA | 1.86 | 1.83 | 1.87 | 1.87 | 1.24 | 1.55 |
| denaturing | content | | | | | | |
| oligo-dT | (mg/mL) | | | | | | |
| | Purity by | 87.3% | 82.0% | 87.3% | 93.7% | 90.9% | 87.7% |
| | FA-CE | | | | | | |
| | (main, pre-, | | | | | | |
| | post-) | | | | | | |
| | Purity by | 93.3% | 88.3% | 90.3% | 95.3% | 88.5% | 88.2% |
| | HPLC (Size- | | | | | | |
| | based) | | | | | | |
| | % Poly A | 97.7% | 96.6% | 99.6% | 99.3% | 99.3% | 99.6% |
| | tail by | | | | | | |
| | HPLC | | | | | | |
| | (Tailed/ | | | | | | |
| | Tailless) | | | | | | |
| | Endotoxin | 0.407 | 0.357 | 0.217 | 0.338 | 0.169 | 0.152 |
| | (EU/mL) | | | | | | |
| | Residual | 0.5 | 0.73 | 0.18 | 3.83 | 0.12 | 0.31 |
| | plasmid | | | | | | |
| | (ng/mL) | | | | | | |
| | Residual | <LOD | <LOD | <LOD | <LOD | <LOD | <LOD |
| | protein by | | | | | | |
| | NanoOrange | | | | | | |

The purification process including HIC followed by denaturing oligo-dT (ddT) was repeated using six further constructs of different lengths: 2872 nt, 2852 nt, 692 nt, 2399 nt, 1772 nt, and 1007 nt. The results showed that the combination of HIC and ddT resulted in high purity, particularly for the longer constructs, with 95-99% poly-A tailed mRNA following ddT (See, Tables 7 and 8).

TABLE 7
Downstream Purification: Percent Tailed mRNA

| | mRNA Integrity Tail RP | | | mRNA Integrity Length RP | | |
|---|---|---|---|---|---|---|
| Length | qIVT | HIC | ddT | qIVT | HIC | ddT |
| nt | % | % | % | % | % | % |
| 2872 | 74.9 | 78.3 | 96.59 | 91.82 | 94.62 | 97.06 |
| 2852 | 52.07 | 93.32 | 97.6 | 93.16 | 94.97 | 96.46 |
| 692 | 94.22 | 96.22 | 99.29 | 96.87 | 94.12 | 97.17 |
| 2399 | 78.45 | 84.71 | 98.07 | 95.31 | 95.37 | 95.61 |
| 1772 | 83.21 | 87.57 | 97.45 | 91.11 | 90.22 | 95.53 |
| 1007 | 81.18 | 90.05 | 98.95 | 92.21 | 90.21 | 96.7 |

TABLE 8
Analytical Panel: Downstream Purification Methods

| | Test | 2872 nt | 2852 nt | 692 nt | 2399 nt | 1772 nt | 1007 nt |
|---|---|---|---|---|---|---|---|
| Test Method | Method | 1.63 | 2.10 | 1.83 | 2.05 | 2.04 | 1.83 |
| Name | ID | mg/mL* | mg/mL* | mg/mL* | mg/mL* | mg/mL* | mg/mL* |
| Identity (Sanger) | TM-25- | Conforms | Conforms | Conforms | Conforms | Conforms | Conforms |
| | 01 | | | | | | |
| Appearance | DSAD- | Clear, | Clear, | Clear, | Clear, | Clear, | Clear, |
| | TM- | colorless | colorless | colorless | colorless | colorless | colorless |
| | 0002 | solution | solution | solution | solution | solution | solution |
| | | no visible | no visible | no visible | no visible | no visible | no visible |
| | | particulates | particulates | particulates | particulates | particulates | particulates |
| PH | DSAD- | 6.5 | 6.7 | 6.6 | 6.5 | 6.6 | 6.5 |
| | TM- | | | | | | |
| | 0009 | | | | | | |
| Total RNA | DSAD- | 1.84 | 2.49 | 2.15 | 2.49 | 2.57 | 2.11 |
| content (mg/mL) | TM- | | | | | | |
| | 0019 | | | | | | |
| Purity by FA- | DSAD- | 9.0, 86.8, | 10.0, 88.3, | 7.6, 89.7, | 8.7, 87.3, | 5.1, 91.5, | 3.2, 95.1, |
| CE (Pre-peak, | TM- | 4.4 | 1.8 | 2.7 | 4.1 | 3.4 | 1.7 |
| Main peak, post- | 0010 | | | | | | |
| peak) | | | | | | | |
| Purity by Size- | DSAD- | 2.0, 90.9, | 5.8, 92.6, | 8.1, 89.4, | 6.4, 92.2, | 4.6, 94.1, | 3.2, 95.3, |
| based HPLC | TM- | 1.1 | 1.6 | 2.5 | 1.4 | 1.3 | 1.5 |
| (Pre-peak, Main | 0026 | | | | | | |
| peak, post-peak) | | | | | | | |
| % Poly A tail by | DSAD- | 97.1 (2.9) | 97.6 (2.4) | 99.6 (0.4) | 96.1 (3.9) | 97.6 (2.4) | 99.2 (0.8) |
| Tailed/Tailless | TM- | | | | | | |
| HPLC (Main | 0035 | | | | | | |
| peak, (pre-peak)) | | | | | | | |
| % 5' Cap by | DSAD- | >99% | >99% | 97% | >99% | >99% | 97% |
| LC/MS | TM- | Capped | Capped | Capped | Capped | Capped | Capped |
| | 0021 | (>99% | (>99% | (95% Cap) | (>99% | (98% Cap) | (97% Cap) |
| | | Cap) | Cap) | | Cap) | | |
| Endotoxin | DSAD- | <0.050 | <0.050 | <0.050 | <0.050 | < .050 | <0.050 |
| (EU/mL) | TM- | | | | | | |
| | 0025 | | | | | | |
| Residual | Real | 0.23 | 0.10 | 0.54 | 1.69 | 0.22 | 1.03 |
| plasmid (ng/mL) | time | | | | | | |
| | qPCR | | | | | | |
| Residual protein | DSAD- | <LOD | <LOD | <LOD | <LOD | <LOD | <LOD |
| by NanoOrange | TM- | (<0.29 | (<0.29 | (<0.29 | (<0.29 | (<0.29 | (<0.29 |
| | 0025 | μg/mL) | μg/mL) | μg/mL) | μg/mL) | μg/mL) | μg/mL) |

A yet additional experiment demonstrated that the combination of HIC and denaturing oligo-dT can be scaled up to large volumes (1 liter column using a 2545 nt mRNA produced by IVT reaction). As shown in Table 9, the purification process provided high percent poly-A tailed mRNA purity (as assessed by both Tris-RP and MP methods).

TABLE 9
HIC/ddT Purification Scale-up

| Chrome | Load | Loading | | % MP |
|---|---|---|---|---|
| Step and | Challenge | Residence | % Tailed | (Size- |
| Cycle | (mg/mL) | Time (min) | (Tris-RP) | based-RP) |
| HICC1 | 4.8 | 5 | 86.9 | 84.2 |
| HICC2 | 5 | 5 | 87.3 | 83.7 |
| HICC3 | 5.5 | 5 | 86.9 | 85.1 |
| HICC4 | 6 | 5 | 87.2 | 85.5 |
| HICC5 | 7.2 | 5 | 87.7 | 84.8 |
| HIC Average | 5.7 | 5 | 87.2 | 84.7 |
| ddT C1 | 3.5 | 20 | 96.9 | 90.0 |
| ddT C2 | 3.5 | 20 | 97.5 | 91.9 |
| ddT C3 | 3.5 | 10 | 97.0 | 91.8 |
| ddT C4 | 3 | 20 | 97.4 | 92.7 |
| ddT C5 | 3 | 10 | 97.5 | 92.8 |
| ddT Average | 3.3 | 16 | 97.3 | 91.8 |

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between the upper and lower ends of the range are specifically contemplated and described herein.

1. A method comprising:
(a) desalting a mixture comprising ribonucleic acid (RNA) to produce a low-salt RNA composition having a salt concentration of less than 20 mM;
(b) heating the low-salt RNA composition to a temperature of higher than 60° C. to produce denatured RNA;
(c) in-line mixing the composition comprising denatured RNA with a high-salt buffer to produce a composition comprising denatured RNA and salt at a concentration of at least 50 mM;
(d) binding the denatured RNA of the composition produced in (c) to an oligo-dT resin at a temperature of lower than 40° C.; and
(e) eluting RNA from the oligo-dT resin.

2. A method comprising:
(a) desalting a mixture comprising ribonucleic acid (RNA) to produce a low-salt RNA composition having a conductivity of less than 2 mS/cm;
(b) heating the low-salt RNA composition to a temperature of higher than 60° C. to produce denatured RNA;
(c) in-line mixing the composition comprising denatured RNA with a high-salt buffer to produce a composition comprising denatured RNA and a conductivity of at least 5 mS/cm;
(d) binding the denatured RNA of the composition produced in (c) to an oligo-dT resin at a temperature of lower than 40° C.; and
(e) eluting RNA from the oligo-dT resin.

3. The method of claim 1 or 2, wherein the mixture of (a) is an in vitro transcription reaction.

4. The method of any one of claims 1-3, wherein the salt of (a) and/or (c) comprises NaCl.

5. The method of any one of claims 1-4, wherein the high-salt buffer has a salt concentration of 100 mM to 1000 mM and/or a conductivity of 5 mS/cm to 85 mS/cm.

6. The method of any one of claims 1-5, wherein the desalting comprises binding the RNA to a hydrophobic interaction chromatography (HIC) resin and eluting the RNA from the HIC resin to produce the low-salt RNA composition.

7. The method of any one of claims 1-6, wherein the low-salt RNA composition has a salt concentration of 1 to 20 mM and/or a conductivity of 0.1 to 2 mS/cm.

8. The method of claim 6, wherein the HIC resin is a (poly)styrene-divinylbenzene (PS-DVB) R150 bead resin with 2000 Angstrom pores.

9. The method of any one of claims 1-8, wherein the heating of (b) occurs for less than 1 minute.

10. The method of any one of claims 1-9, wherein the heating of (b) occurs for at least 10 seconds.

11. The method of any one of claims 1-10, wherein the heating of (b) occurs for 10 to 60, 10 to 30, 20 to 40, or 30 to 60 seconds.

12. The method of any one of claims 1-11 wherein the heating of (b) occurs at a temperature of 60° C. to 90° C.

13. The method of any one of claims 1-12, wherein the low-salt RNA composition is heated in the presence of a denaturant molecule.

14. The method of claim 13, wherein the denaturant molecule is dimethyl sulfoxide, guanidine, or urea.

15. The method of any one of claims 1-14 further comprising, between (b) and (c), in-line cooling the composition comprising denatured RNA to a temperature of lower than 60° C.

16. The method of any one of claims 1-15 further comprising, between (b) and (c), in-line cooling the composition comprising denatured RNA to a temperature of lower than 40° C.

17. The method of any one of claims 1-16 further comprising, between (b) and (c), storing the composition comprising denatured RNA in a break tank.

18. The method of claim 17, wherein the composition comprising denatured RNA is stored in the break tank for 1 to 5 days at 2 to 8° C.

19. The method of any one of claims 1-18, wherein the in-line mixing of (c) occurs for less than 1 minute.

20. The method of any one of claims 1-19, wherein the composition comprising denatured RNA produced in (c) has a salt concentration of 50 to 500 mM and/or a conductivity of 5 to 85 mS/cm.

21. The method of any one of claims 1-20, wherein at least 90% of the total RNA in the composition of (c) comprises denatured RNA.

22. The method of any one of claims 1-21, wherein the binding of (d) occurs at a temperature of 4° C. to 25° C.

23. The method of any one of claims 1-22, wherein the binding of (d) occurs for less than 20 minutes.

24. The method of any one of claims 1-23, wherein the oligo-dT resin is a (poly)styrene-divinylbenzene (PS-DVB) bead resin with 2000 Angstrom pores derivatized with poly dT.

25. The method of any one of claims 1-24, wherein the RNA eluted from the oligo-dT resin comprises at least 90% poly-A tailed mRNA.

26. The method of claim 25, wherein the RNA eluted from the oligo-dT resin comprises at least 95% poly-A tailed mRNA.

27. The method of any one of claims 1-26, wherein the in-line mixing occurs concurrent with binding the mixture on the oligo-dT resin.

28. The method of any one of claims 1-27, wherein the secondary structure of the RNA is monitored before and after (b) using ultraviolet detection.

29. The method of any one of claims 1-28, wherein denaturation of the RNA during (b) is monitored using ultraviolet detection.

30. A method comprising:
in-line mixing a high-salt buffer with a low-salt denatured RNA composition that comprises denatured ribonucleic acid (RNA) to produce a high-salt composition comprising denatured RNA; and
binding the denatured RNA to an oligo-dT resin.

31. The method of claim 30, wherein the in-line mixing occurs for less than 1 minute before binding the denatured RNA to the oligo-dT resin.

32. The method of claim 30 or 31, wherein the high-salt buffer has a salt concentration of at least 100 mM and/or a conductivity of at least 10 mS/cm.

33. The method of claim 32, wherein the high-salt buffer has a salt concentration of 100 mM to 1000 mM NaCl.

34. The method of any one of claims 30-33, wherein the low-salt denatured RNA composition has a salt concentration of less than 20 mM and/or less than 2 mS/cm.

35. The method of any one of claims 30-34, wherein the low-salt denatured RNA composition has a salt concentration of 1 to 20 mM and/or a conductivity of 0.1 to 2 mS/cm.

36. The method of any one of claims 30-35, wherein the high-salt composition has a salt concentration of 50 to 500 mM and/or a conductivity of 5 to 85 mS/cm.

37. The method of any one of claims 30-36, wherein the denatured RNA in the high-salt composition makes up at least 90% of total RNA in the high-salt composition.

38. The method of any one of claim 30-37, wherein the binding occurs for less than 20 minutes.

39. The method of any one of claims 30-38, wherein the binding occurs at a temperature of lower than 40° C.

40. The method of claim 39, wherein the binding occurs at a temperature of 4° C. to 25° C.

41. The method of any one of claims 30-40, wherein the oligo-dT resin is a (poly)styrene-divinylbenzene (PS-DVB) bead resin with 2000 Angstrom pores derivatized with poly dT.

42. The method of any one of claims 30-41 further comprising eluting RNA from the oligo-dT resin in a low-salt buffer.

43. The method of claim 42, wherein the RNA eluted from the oligo-dT resin comprises at least 90% poly-A tailed mRNA.

44. The method of claim 43, wherein the RNA eluted from the oligo-dT resin comprises at least 95% poly-A tailed mRNA.

45. The method of any one of claims 30-44, further comprising:
prior to in-line mixing with a high-salt buffer, heating a composition comprising RNA and a salt concentration of less than 20 mM to a temperature of higher than 60° C. to produce a denatured RNA composition.

46. The method of any one of claims 30-44, further comprising:
prior to in-line mixing with a high-salt buffer, heating a composition comprising RNA and having a conductivity of less than 2 mS/cm to a temperature of higher than 60° C. to produce a denatured RNA composition.

47. The method of claim 45 or 46, wherein the heating occurs for less than 1 minute.

48. The method of any one of claims 45-47, wherein the heating occurs for at least 10 seconds.

49. The method of any one of claims 45-48, wherein the heating occurs for 10 to 60, 10 to 30, 20 to 40, or 30 to 60 seconds.

50. The method of any one of claims 45-49, wherein the heating occurs at a temperature of 60° C. to 90° C.

51. The method of any one of claims 30-50, further comprising:
prior to in-line mixing with a high-salt buffer, desalting a mixture comprising RNA to produce an RNA composition having a salt concentration of less than 20 mM; and
heating the RNA composition having a salt concentration of less than 20 mM to a temperature of higher than 60° C. to produce the denatured RNA composition.

52. The method of claim 51, wherein the mixture is an in vitro transcription reaction.

53. The method of claim 51 or 52, wherein the desalting comprises binding the RNA to a hydrophobic interaction chromatography (HIC) resin and eluting the RNA from the HIC resin to produce an RNA composition.

54. The method of claim 53, wherein the HIC resin is a (poly)styrene-divinylbenzene (PS-DVB) R150 bead resin with 2000 Angstrom pores.

55. The method of any one of claims 30-54, wherein the secondary structure of the RNA is monitored using ultraviolet detection during the heating of the composition.

56. The method of any one of claims 30-55, wherein denaturation of the RNA is monitored using ultraviolet detection during the heating of the composition.
